(19) United States
(12) Patent Application Publication
(10) Pub. No.: US 2026/0123963 A1
(43) Pub. Date: May 7, 2026

(54) BONE ANCHOR EXTENDED TAB BRIDGE CUTTING AND TAB BREAKING INSTRUMENT

(71) Applicant: SPINE WAVE, INC., Shelton, CT (US)

(72) Inventors: Eugene Avidano, Stratford, CT (US); David Boisvert, Southington, CT (US); Andrea Wright, Southington, CT (US)

(73) Assignee: SPINE WAVE, INC., Shelton, CT (US)

(21) Appl. No.: 19/369,529

(22) Filed: Oct. 27, 2025

Related U.S. Application Data
(60) Provisional application No. 63/715,739, filed on Nov. 4, 2024.

(51) Int. Cl. A61B 17/70; A61B 17/86

(52) U.S. Cl. CPC A61B 17/7032; A61B 17/866

(57) ABSTRACT

An instrument and method for cutting a pair of bridges connecting opposing extended tabs of a spinal bone anchor yoke receiver. The instrument includes a pair of opposing movable handles pivotally coupled to each other and a pair of opposing movable legs respectively at the distal end of the handles, each leg supporting a cutting blade facing the other cutting blade. A pair of anvil surfaces is disposed between the cutting blades and spaced therefrom for receipt of a respective bridge. Movement of the handles causes each cutting blade to move toward an anvil surface and compress a respective bridge between a respective cutting blade and an anvil surface until each bridge is severed. The method includes attaching the instrument to at least one of the extended tabs, cutting the bridges, and manipulating the instrument back and forth to remove the one extended tab from the yoke receiver.

### CROSS REFERENCE TO RELATED APPLICATIONS

This application claims the benefit of U.S. Provisional Patent Application No. 63/715,739, filed Nov. 4, 2024, the entire contents of which are incorporated by reference herein.

### FIELD OF THE INVENTION

The subject invention relates generally to the field of instruments used in spinal surgery, and more particularly, to an instrument for cutting a bridge joining bone anchor extended tabs and for breaking off one or more extended tabs from the bone anchor upon attachment to a spinal fixation rod.

### BACKGROUND OF THE INVENTION

Spinal fixation devices are commonly used in spinal surgery to hold one or more vertebral motion segments in a fixed position to allow fusion or healing. Such fixation devices commonly include a rigid spinal rod that is joined to adjacent vertebral bodies by attaching the rod to bone anchoring devices, such as hooks and/or bone screws. Bone screws, known as pedicle screws for example, may be configured to be inserted into a pedicle of a vertebral body for securing the spinal rod in the fixation construct.

Such pedicle screws typically comprise a threaded shank for threaded engagement into a pedicle of a vertebral body, and a yoke attached to a head of the threaded shank for receiving a spinal fixation rod. In some instances, the yoke may be polyaxially coupled to the head of the threaded shank, either before or after the threaded shank is introduced into the pedicle. The yoke typically includes a pair of spaced arms defining a U-shaped channel for receipt of the spinal rod. A fastening mechanism, such as a set screw, is commonly used to lock the spinal rod into the receiving channel of the yoke.

With the advent of minimally invasive and percutaneous spinal surgery, a need has developed for the placement of spinal implants, including pedicle screws and fixation rods, though one or more small incisions formed through the skin of the patient from a position outside the patient. This has resulted in the addition of extended tabs that are breakably attached at their distal ends to the respective arms of the yoke, with the extended tabs being of length such that the proximal ends extend through the skin surface of the patient for access by the surgeon. Such extended tabs are commonly attached to the yoke arms at a frangible portion that is configured to break under a predetermined force so that the extended tabs may be separated from the yoke upon completion of the surgery.

The extended tabs are separated by opposed slots that are configured to receive the spinal fixation rod and for guiding the spinal rod down to the U-shaped channel of the yoke. In some instances, the proximal ends of the extended tabs are not coupled, thereby allowing top entry of the spinal rod into the slots in a direction transverse to its longitudinal axis. While this open configuration is conducive to the placement of the spinal rod, the free unattached proximal ends of the extended tabs may undesirably move during surgery reducing the efficiency of the procedure. To provide additional strength and stability to the extended tabs during use, the proximal ends may be coupled by one or more bridges. Since the bridges at the proximal end close off a top entry of the rod into the slots the spinal rod has to be introduced into the slots between the extended tabs along the longitudinal axis of the rod.

While extended tabs coupled with a bridge provide beneficial strength and stability, in order to break the extended tabs off the yoke at the frangible portions at the distal ends, the bridge needs to be severed, so that the respective extended tabs may be individually removed from the yoke. Accordingly, there is a need for an instrument that can desirably cut or fracture one or more bridges that couple extended tabs, as well as to facilitate and/or participate in the breaking of extended tabs from the yoke of the bone anchor.

### SUMMARY OF THE INVENTION

It is an object of the subject invention to provide an improved instrument for cutting a pair of bridges that connect opposing extended tabs extending from a yoke receiver of a spinal bone anchor.

It is another object of the invention to provide an improved method of cutting a pair of bridges connecting a pair of opposing extended tabs extending from a yoke receiver of a spinal bone anchor, the bridges being disposed at a proximal end of the extended tabs, and for breaking off the extended tabs from the spinal bone anchor that are connected to the yoke receiver by frangible connectors at a distal end of the extended tabs.

### BRIEF DESCRIPTION OF THE FIGURES

FIG. 1 is a perspective view of an instrument positioned for cutting bridges connecting a pair of opposing extended tabs extending from a modular yoke attachable to a bone anchor.

FIG. 2 is an enlarged view of the encircled portion A of FIG. 1.

FIG. 3 is an unexploded perspective view of components of the cutting instrument of FIG. 1.

FIG. 4 is an elevation view of the cutting instrument of FIG. 1.

FIG. 5 is a cross-sectional view of the cutting instrument of the subject invention as seen along viewing lines A-A of FIG. 4.

FIG. 6 is an elevation view showing the cutting instrument of the subject invention as used to break an extended tab from the modular yoke after the joining bridges have been severed.

### DETAILED DESCRIPTION OF THE EMBODIMENTS

For the purposes of promoting and understanding of the principles of the invention, reference will now be made to the embodiments illustrated in the drawings and described in the following written specification. It is understood that no limitation to the scope of the invention is thereby intended. It is further understood that the present invention includes any alterations and modifications to the illustrated embodiments and includes further applications of the principles of the invention as would normally occur to one skilled in the art to which this invention pertains.

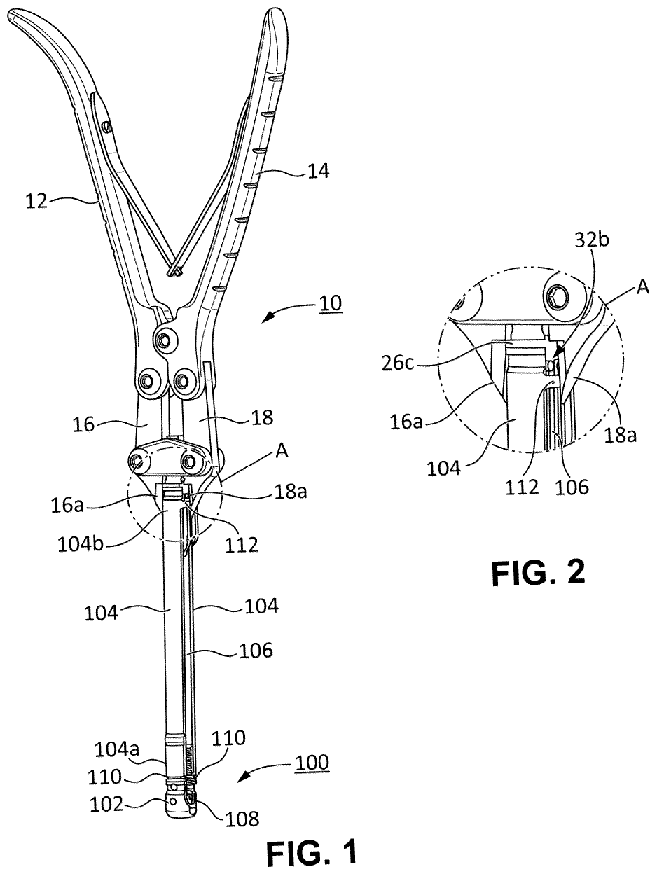

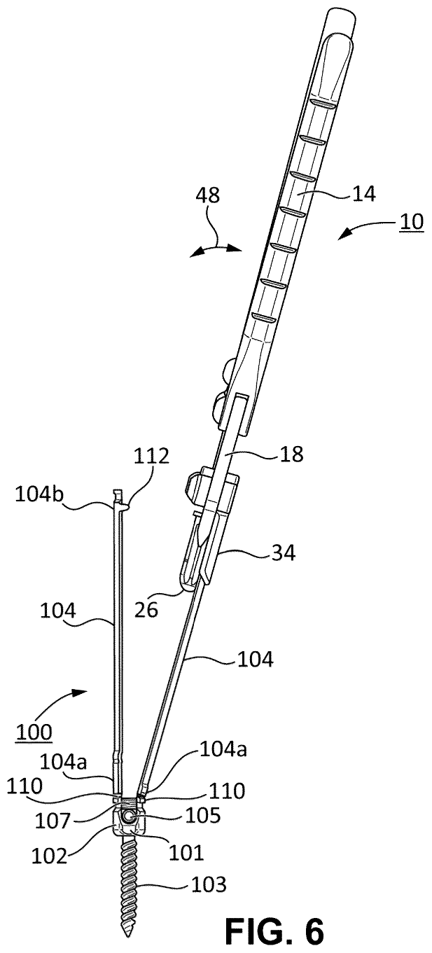

Referring to FIGS. 1 and 6, a cutting instrument 10 is shown in position for cutting bridges connecting a pair of opposing extended tabs extending from a modular yoke 100 that is attachable to a bone anchor, such as a pedicle screw 103, in spinal surgery. As depicted, modular yoke 100 includes a yoke receiver 102 comprising a pair of spaced opposing extended tabs 104 that together define a slot 106 for receipt of a spinal rod 105. Yoke receiver 102 has a pair of spaced arms defining a U-shaped channel 108 in communication with slot 106 for receipt and seating of the spinal rod 105. Tabs 104 are attached to a yoke receiver 102 at their distal ends 104a by frangible connectors 110 that may be broken to separate arms 104 from receiver 102 after proper seating of the spinal rod 105 into the receiver channel 108. Yoke receiver 102 has an opening at its lower distal end for receipt of a head 101 of a pedicle screw 103. It should be understood that modular yoke 100 may be pre-assembled to pedicle screw 103 prior to introduction of pedicle screw 103 into a vertebral body of a patient or attached after pedicle screw 103 is inserted.

At the upper proximal end 104b, opposing tabs 104 are connected by a pair of bridges 112, one such tab 112 being shown in further detail in FIG. 2. The additional length of the construct provided by extended tabs 104 assists in the receipt of the spinal rod 105 in surgery and for guiding the spinal rod 105 down into receiver 102 for proper seating prior to being secured by a fastening screw. The length of tabs 104 is configured such that during surgery proximal ends 104b extend exteriorly from the outer surface of the skin of a patient when yoke 100 is attached to a pedicle screw 103 installed in a vertebral body of a patient. Such a construction allows for use in minimally invasive spinal procedures wherein a separate individual incision may be provided for each pedicle screw 103. Further details of a modular yoke receiver with extended tabs are described in commonly assigned U.S. Pat. No. 11,523,850, entitled “Modular Spinal Screw Yoke Inserter”, issued to Eugene Avidano et al. on Dec. 13, 2022, the contents of which are incorporated herein by reference in their entirety.

Upon satisfactory introduction and seating of a spinal rod 105 in yoke receiver 102 and securement thereto by a fastening mechanism, such as a set screw 107 (see FIG. 6), the extended yokes 104 must be separated from the yoke receiver 102 to allow the surgeon to complete the spinal surgery. Because bridges 112 provide strength and stability to the extended tab structure, in order to break extended tabs 104 off the yoke receiver 102 at the frangible connectors 110, the bridges 112 need to be severed initially so that the respective extended tabs 104 may be removed from the yoke receiver 102. Cutting instrument 10 is provided for such purpose.

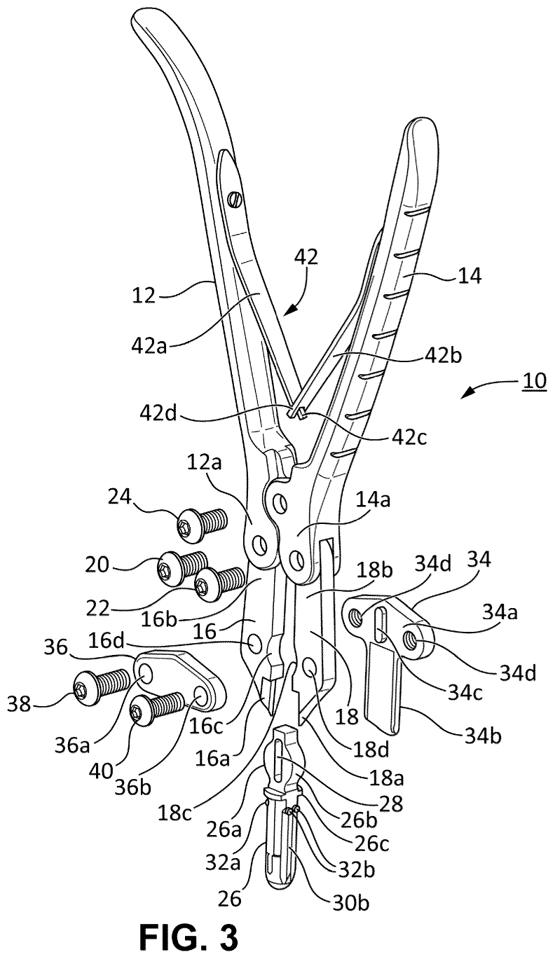

Turning now to FIGS. 3-5, details of cutting instrument 10 are described. Cutting instrument comprises a pair of handles 12 and 14 that are pivotably coupled to each other, as will be described. The distal ends 12a and 14a of each handle 12 and 14 are pivotably coupled, respectively to a pair of the legs 16 and 18, that terminate in cutting blades 16a and 18a, respectively. Upper end 16b of leg 16 is pivotally coupled to distal end 12a of handle 12 by threaded fastener 20 while the upper end 18b of leg 18 is pivotally coupled to distal end 14a of handle 14 by a threaded fastener 22. Similarly, handles 12 and 14 are pivotally coupled to each other by a threaded fastener 24, although it should be understood that other fastening elements, such as rivets, pins or welds may also be used. A pair of curved surfaces 16c and 18c are formed, respectively, into legs 16 and 18 in opposed facing relation to each other.

As illustrated in FIG. 3, cutting instrument 10 further includes an elongate guide pin 26 that is configured to be received, in part, between legs 16 and 18. Guide pin 26, in one arrangement, has a solid cross-section as seen in FIG. 5. An elongated vertical slot 28 is formed through the upper end of guide pin 26. A pair of curved surfaces 26a and 26b is formed on opposite sides of guide pin 26 adjacent slot 28. Curved surfaces 26a and 26b are configured to be received into curved surfaces 16c and 18c of legs 16 and 18, respectively. Guide pin curved surfaces 26a and 26b are formed to have a radius of curvature slightly less than the radius of curvature of curved surfaces 16c and 18c of legs 16 and 18 to allow slight linear movement of guide pin 26 between legs 16 and 18, as will be described. Extending from approximately the center of guide pin 26 to a location near the bottom of guide pin 26, are a pair of opposing, elongated, relatively pointed edges 30a and 30b, as also shown in FIG. 5. Each pointed edge 30a and 30b defines an anvil that cooperates with blades 16a and 18a for cutting extended tab bridges 112, as will be described. Cutting blade alignment structure, such as a pair of alignment pins 32a and 32b (see also FIG. 2) are formed on opposite sides of elongated edges 30a and 30b to maintain cutting blades 16a and 18a in a desirable orientation directly opposite edges 30a and 30b for severing bridges 112. Guide pin 26 is preferably formed to have an outer diameter that approximates the inner diameter defined by opposing extended tabs 104. Below slot 28 and above alignment pins 32a and 32b, guide pin 26 includes, in one arrangement, an outwardly projecting flange 26c that defines a stop surface, as will be described.

Referring still to FIG. 3, cutting instrument 10 includes a support plate 34 and a front plate 36 that are secured to each other and to legs 16 and 18. Support plate 34 has an upper flange 34a and a lower support surface 34b depending downwardly therefrom. An elongated, vertically extending rail 34c is formed generally centrally through flange 34a. Rail 34c projects outwardly from flange 34a toward legs 16 and 18 and is configured to be received within the slot 28 of guide pin 26. Rail 34c has a length slightly less than a length of slot 28 to thereby allow linear movement of guide pin 26 relative to support plate 34. Support surface 34b is curved outwardly and has a radius of curvature slightly larger than a radius of curvature of an outer surface of extended tab 104. Support surface 34b has a length slightly less than the length of guide pin 26. A pair of threaded holes 34d and 34e is formed through flange 34a on either side of rail 34c.

Front plate 36 has a pair of spaced clearance holes 36a and 36b formed therethrough. A threaded fastener 38 is provided to extend through clearance hole 36a and through a clearance hole 16d formed through leg 16 for threaded engagement into threaded hole 34d of support plate 34. A threaded fastener 40 is provided to extend through clearance hole 36b and through a clearance hole 18d formed through leg 18 for threaded engagement into threaded hole 34e of support plate 34. Leg clearance holes 16d and 18d are configured to be slightly larger than the outer threaded diameter of fasteners 38 and 40 so as to allow slight movement of legs 16 and 18 toward and away from each other relative to support plate 34 and front plate 36 during operation. Upon attachment of support plate 34 and front plate 36 to legs 16 and 18, curved support surface 34b is spaced radially from guide pin 26 by a space, S, as shown in FIG. 5. Space, S is configured to be slightly greater than the thickness of a wall of an extended tab 104 such that the extended tab 104 can be closely captured between guide pin 26 and curved surface 34b of support plate 34.

A spring 42 is provided between handles 12 and 14. Spring 42 is of conventional construction having a first cantilevered spring member 42a and a second cantilevered spring member 42b. First spring member 42a is suitably attached to handle 12 and second spring member 42b is suitably attached to handle 14. Free ends 42c and 42d of respective spring members 42a and 42b cooperate to provide bias to handles 12 and 14 in a manner to normally maintain blades 16a and 18s in an open position before use.

All of the components of cutting instrument 10,/including the screws may be formed of stainless steel, although other suitable metallic materials, such as titanium or aluminum may be used. In a particular arrangement, legs 16 and 18 that include cutting blades 16a and 18a, respectively, may be formed of a hardened stainless steel, such as SST 465 H950.

Having described the details of cutting instrument 10, operation and use of cutting instrument 10 are now described. Referring now to FIGS. 1, 2 and 4, the initial placement of cutting instrument 10 onto extended tabs 104 of yoke receiver 102 is described. As shown, cutting blades 16a and 18a are placed in an open position that will allow receipt therebetween of the proximal ends 104b of extended tabs 104, including bridges 112 that join opposing tabs 104. Guide pin 26 is introduced into the extended tab structure between opposing extended tabs 104 until flange 26c abuts the proximal end of extended tabs 104, as particularly shown in FIG. 2. In this position, cutting blades 16a and 18a are in juxtaposition with bridges 112 with the proximal end 104b of one extended tab 104 being disposed in space, S between the outer surface of guide pin 26 and the inner surface of the support surface 34b of support plate 34.

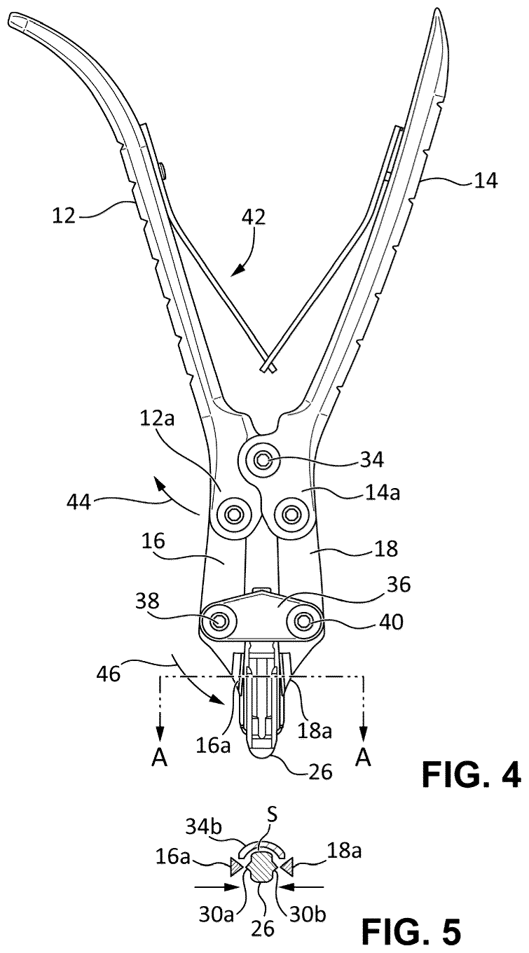

At this point, handles 12 and 14 are manually squeezed toward each other to overcome the bias of spring 42, causing the handle distal ends 12a and 14a to pivot outwardly about threaded fasteners 20, 22 in the direction as shown by arrow 44 in FIG. 4. This in turn will cause the lower ends of legs 16 and 18 to pivot inwardly toward each other, as shown by arrow 46, about fastening elements 38 and 40 causing blades 16a and 18a to engage the outer surfaces of bridges 112. Continued manual squeezing of handles 12 and 14 will cause bridges 112 to be compressed between cutting blades 16a, 18a and opposing anvil surfaces provided by edges 30a, 30b of guide pin 26. Handles 12 and 14 are further squeezed until the compression force is sufficient to sever the bridges. During this cutting action, there is slight upward movement of guide pin 26 between legs 16, 18 along rail 34c as the curved surfaces 26a and 26b of guide pin 26 engage the slightly larger curved surfaces 16c, 18c of legs 16 and 18, respectively. Such movement of guide pin 26 relative to legs 16, 18 will tend to provide a slicing action between cutting blades 16a, 18a and edges 30a, 30b that contributes to the cutting of the bridges 112.

With bridges 112 severed, extended tabs 104 may now be separated from yoke receiver 102, as illustrated in FIG. 6. After bridges 112 are cut, cutting instrument 10 may remain attached to one extended tab 104, with the proximal end 104b of such one extended tab 104 being frictionally held between guide pin 26 and support surface 34b of support plate 34. Cutting instrument 10 may then be used to work the extended tab 104 back and forth as shown by arrow 48 about frangible connector 110. Such back and forth action will ultimately break the weakened portion of the frangible connector 110, thereby separating extended tab 104 from yoke receiver 102. During such back and forth movement, cutting instrument 10 may be moved a sufficient distance to engage the opposing extended tab 104 to thereby contribute to the weakening of the frangible connector 110 attaching such opposing extended tab to yoke receiver 102 and allowing easy removal of the opposing extended tab 104. With both extended tabs 104 removed. From yoke receiver 102 the surgeon may complete the surgical procedure. As such, it should be appreciated that cutting instrument 10 may be used to both cut bridges 112 joining proximal ends of extended tabs 104 and break one or more frangible connections that join the extended tabs 104 to the yoke receiver 102.

While the invention has been illustrated and described in detail in the drawings and foregoing description, the same should be considered as illustrative and not restrictive in character. Accordingly, it is understood that only the preferred embodiments have been presented and that all changes, modifications and further applications that come within the spirit of the invention are desired to be protected.

What is claimed is:

1. An instrument for cutting a pair of bridges connecting a pair of opposing extended tabs extending from a yoke receiver of a spinal bone anchor, said opposing extended tabs defining a slot therebetween for receipt of a spinal rod, said instrument comprising:
a pair of opposing movable handles pivotally coupled to each other, said handles each having a proximal end and a distal end:
a pair of opposing movable legs, one leg each being pivotally coupled to a respective handle at its distal end thereof;
a pair of cutting blades, one blade being supported on each of said legs in facing relation to the other cutting blade; and
a guide pin supported by said legs and disposed between said cutting blades, said guide pin including a pair of anvil surfaces, each one of said anvil surfaces facing a respective one of said cutting blades and spaced therefrom for receipt of a respective bridge,
wherein movement of said handles causes each of said cutting blades to move toward said guide pin and compress a respective bridge between a respective cutting blade and an anvil surface until each bridge is severed.

2. The instrument of claim 1, wherein a support plate is attached to each leg in a manner to allow relative movement of said legs toward and away from each other.

3. The instrument of claim 2, wherein said support plate has an outwardly curved inner surface facing said guide pin, said curved inner surface having a radius of curvature slightly larger than a radius of curvature of an outer surface of one of said extended tabs to define a space therebetween for receipt of said one extended tab.

4. The instrument of claim 3, wherein said guide pin is movable linearly relative to said legs in a direction toward and away from said handles.

5. The instrument of claim 4, wherein said support plate includes a vertically extending elongated rail, and wherein said guide pin has an elongated vertically extending slot sized and configured for linear movable receipt of said rail.

6. The instrument of claim 5, wherein said guide pin includes a pair of outwardly projecting curved surfaces on opposite sides of guide pin adjacent said elongated slot, and wherein each leg comprises an inwardly directed curved surface more proximal than said cutting blades for receipt therein of said outwardly projecting curved surfaces of guide pin.

7. The instrument of claim 6, wherein said inwardly directed curved surfaces each have a radius of curvature slightly larger than a radius of curvature of said outwardly projecting curved surfaces to allow slight linear movement of said guide pin between said legs.

8. The instrument of claim 7, wherein each of said anvil surfaces is defined by elongated edges projecting outwardly from opposite sides of said guide pin.

9. The instrument of claim 8, wherein said guide pin further includes a pair of alignment pins more proximal than and on opposite sides of elongated edges.

10. The instrument of claim 9, wherein said guide pin further includes an outwardly projecting flange between said elongated slot and said alignment pins that defines a stop surface that is sized and configured to abut a proximal end of said extended tabs to position said cutting blades in juxtaposition with said respective bridges.

11. The instrument of claim 10, wherein said guide pin has an outer diameter more distal than said alignment pins that approximates an inner diameter defined by said opposing extended tabs.

12. The instrument of claim 11, further including a spring disposed between said handles to bias said handles so that said cutting blades are in a normally open position.

13. A method of cutting a pair of bridges connecting a pair of opposing extended tabs extending from a yoke receiver of a spinal bone anchor, said bridges being disposed at a proximal end of said extended tabs, and for breaking off said extended tabs from said spinal bone anchor that are connected to said yoke receiver by frangible connectors at a distal end of said extended tabs, comprising the steps of:
attaching an instrument to at least one of said extended tabs, said instrument comprising an operable pair of opposing cutting blades;
operating said cutting blades to cut both of said bridges to thereby separate said extended tabs from each other at said proximal end; and
with said instrument remaining attached to said one extended tab manipulating said instrument back and forth until said one extended tab is removed from said yoke receiver.

14. The method of claim 13, wherein during said manipulating step, the method further includes the step of engaging said opposing extended tabs with said instrument to weaken the frangible connector attaching said opposing extended tab to said yoke receiver to thereby allow manual removal of said opposing extended tab.

15. The method of claim 13, including the step of providing said instrument with a pair of anvil surfaces opposing said cutting blades, and placing said instrument on said one extended tab such that each of said bridges is respectfully placed between a cutting blade and an anvil surface.

16. The method of claim 15, wherein said instrument is placed onto the proximal ends of said extended tabs with said cutting blades being located in juxtaposition exteriorly of said bridges and said anvil surfaces being located in juxtaposition interiorly of said bridges.

17. The method of claim 16, wherein said operating step is practiced by moving said cutting blades toward each of said respective bridges to compress said bridges between said respective anvil surfaces and said cutting blades until said bridges are severed.

18. The method of claim 17, wherein said instrument comprises a stop surface for engaging the proximal ends of said extended tabs, and wherein during the attachment step said stop surface is placed in an engagement with said proximal ends to place said anvil surfaces and said cutting blades in axial juxtaposition with said bridges.

19. The method of claim 18, wherein said instrument comprises alignment pins, and wherein said alignment pins are placed during the attachment step of said instrument onto said extended tabs to radially align said cutting blades and said anvil surfaces with said bridges.

20. The method of claim 19, wherein said instrument comprises a guide pin that is supported between said cutting blades, said guide pin including said stop surface said alignment pins, and wherein said instrument further includes a support plate spaced from said guide pin to define a space therebetween, and wherein during the attachment step said one extended tab is placed into said space.
